(19) United States
(12) Patent Application Publication
(10) Pub. No.: US 2014/0322324 A1
(43) Pub. Date: Oct. 30, 2014

(54) Composition For The Treatment Of Bacterial Infections

(71) Applicant: Georgios PANDALIS, Glandorf (DE)

(72) Inventor: Georgios Pandalis, Glandorf (DE)

(21) Appl. No.: 14/358,083

(22) PCT Filed: Dec. 14, 2012
(86) PCT No.: PCT/EP2012/005170

§ 371 Date: May 14, 2014

Related U.S. Application Data
(60) Provisional application No. 61/570,338, filed on Dec. 14, 2011.

(30) Foreign Application Priority Data
Dec. 14, 2011 (EP) 11009852.2

(51) Int. Cl. A61K 36/53; A61K 36/185

(52) U.S. Cl. CPC A61K 36/53; A61K 36/185
USPC 424/479; 424/474; 424/745

(57) ABSTRACT

Composition for use in treating bacterial infections, comprising a concentrate or an extract of origanum in combination with at least one concentrate or extract of another plant.

The present invention relates to a composition comprising a concentrate or an extract of origanum for the treatment of bacterial infections.

A great number of diseases are caused by bacterial infection. The fight against bacterial infection represents one of the high points in modern medicine. The development of antibiotics in the 1940s offered physicians a powerful tool against bacterial infections that has saved the lives of millions of people. However, because of the widespread and sometimes inappropriate use of antibiotics, strains of bacteria that are antibiotica-resistant have begun to emerge. These new, stronger bacteria pose a significant threat to general welfare and health.

Bacterial infections can be caused by a wide range of bacteria, resulting in mild to life-threatening illnesses (such as bacterial meningitis) that require immediate intervention. Common bacterial infections include for example pneumonia, ear infections, diarrhea, urinary tract infections, and skin disorders.

The treatment of bacterial infections is most often achieved by using antibiotics which either aim at killing invading bacteria (bactericide mode of action) or inhibiting their growth (bacteriostatic mode of action) without harming the host. Antibiotic effectiveness depends on mechanism of action, drug distribution, site of infection, immune status of the host, and resistance factors of bacteria. Antibiotics work through several mechanisms; some inhibit the formation of bacterial cell walls. Others interrupt bacterial protein synthesis. Yet some others inhibit metabolism or interfere with DNA synthesis and/or cell membrane permeability.

Since their discovery in the 1940s, many antibiotics have lost their effectiveness against common bacterial infections since bacteria developed resistances against the drugs leading to increased hospitalizations, health costs, and mortality.

In particular, Staphylococcus aureus, a facultative anaerobic Gram-positive coccal bacterium, is considered as one of the most dangerous microorganisms of our days by virtue of its resistance against to all common antibiotics including for example vancomycin. S. aureus can cause a range of diseases ranging from minor skin infections, such as pimples, impetigo, boils, cellulitis folliculitis, carbuncles, skaled skin syndrome, and abscesses, to life-threatening diseases such as pneumonia, meningitis, osteomyelitis, endocarditis, toxic shock syndrome, bacteraemia, and sepsis. Its incidence is from skin, soft tissue, respiratory, bone, joint, endovascular to wound infections. In particular, methicillin-resistant S. aureus (MRSA) is troublesome since these organisms have developed resistances not only to β-lactams but also to many other antimicrobial agents. MRSA is most often found in hospitals and nursing homes, where patients with open wounds, invasive devices, and weakened immune systems are at greater risk of infection than the general public.

An alternative approach for the treatment of bacterial infections consists in the use of bacteriostatic nutrients. Origanum, for example, has been known for many centuries for its antibacterial activity.

Origanum essential oil has been used for a long time in far Eastern and Middle Eastern cultures to treat respiratory infections, chronic inflammation, urinary tract infections, dysentery, and jaundice. A large number of in vitro studies have shown origanum oil, or its most active constituents carvacrol and thymol, to inhibit the growth of a broad range of bacteria.

Hammer and colleagues for example investigated 52 plant oils for activity against nine bacteria. Origanum oil was one of only three oils that inhibited the growth of Pseudomonas aeruginosa, a hard-to-kill bacterium that causes human wound infections. Overall, origanum oil was better at inhibiting germ growth than all oils tested except lemon grass oil (Hammer, K. et al. J. Appl. Microbial 1999, 86, 985-990).

Baretta and coworkers tested origanum oil besides sage, rosemary, laurel and coriander oils against 25 bacteria. They noted that origanum oil manifested the broadest and highest activity against almost all of the bacteria tested; in fact it strongly inhibited 19 of the 25 bacterial strains under investigation (Baretta, M. T. et al. J. Essent. Oil Res. 1998, 10, 618-627).

In a variety of studies, it has been demonstrated that origanum oil provides efficient activity in the inhibition of growth of S. aureus.

Celik and co-workers for example investigated the antimicrobial activity of the essential oil of Origanum hypericifolium against a number of Staphylococcus aureus strains including MRSA strains. Results showed that O. hypericifolium has the potential for being used in food and medicine because of its antibacterial and antioxidant activity (Celik, A.; Nur Herken, E.; Arslan, I.; Zafer Ozel, M.; Mercan, N. Nat. Prod. Res. 2010, 24, 1568-1577).

Another study investigated the antibacterial activity of the essential oil in comparison to infusion, and decocation derived from Origanum vulgare against 111 gram-positive bacterial isolates belonging to 23 different species related to 3 genera. While essential oil and infusion exhibited antibacterial activity against S. saprophyticus and S. aureus inter alia, the isolates were found to be resistant to decoction of origanum (Saeed, S.; Tariq, P; Pak. J. Pharm. Sci. 2009, 22, 421-424).

The antibacterial effects of origanum oils were also demonstrated in in vivo studies carried out with mice. Origanum oil was shown to be bactericidal in culture to two strains of Staphylococcus aureus (ATCC #14154 and #14775) at 0.25 mg/mL. In vitro, monolaurin's effects mirrored origanum oil. The combination of both was bactericidal at the 0.125 mg/mL concentration of each. In two separate in vivo experiments, injected S. aureus (ATCC #14775) killed all 14 untreated mice within a 1-week period. In treated mice, over one third survived for 30 days when given oral origanum oil daily for 30 days (6/14). Over 60% of mice survived when receiving a daily combination of origanum oil and monolaurin (5/8). This study shows that origanum oil in combination with monolaurin may prove to be a useful antimicrobial agent for prevention and therapy of S. aureus infections.

Most of the studies investigating the antibacterial properties of origanum use the essential oil obtained by distillation. An extract obtained by dissolving origanum leaves in ethanol has been described by Yoshino and coworkers, who investigated the antioxidant and antiflammatory activities of the origanum extract. However, this study does not relate to the antibacterial properties of origanum (Yoshino K.; Higashi, N., Koga, K. J. of Health Sci. 2006, 52, 169-173).

Further investigations focus on the essential oil of origanum obtained by solvent-free microwave extraction (SFME), supercritical fluid extraction or hydrodistillation. A study conducted by Karakaya et al. revealed that essential oils obtained by solvent-free microwave extraction at different microwave powers and hydrodistillation inhibited the survival of Listeria monocytogenases, Salmonella typhimurium, and Escherichia coli O157.H7, whereas survival of Staphylococcus aureus was not influenced.

The above described studies illustrate the dependency of the antimicrobial activity of origanum on the procedure the active compounds are isolated from the plant. According to most of the studies performed, essential origanum oil obtained by distillation seems to provide the greatest potential for inhibiting bacterial growth.

A different strategy to enhance the efficacy of the activity profile of a medical plant consists in the combination of different herbs so as to enhance the efficacy synergistically. For example, WO-A-2010091415 describes an antimicrobial composition containing low concentrations of essential oils and a botanical extract in combination with a fruit acid and alkanediol, and optionally a solvent. This invention, however, is silent about the use of an origanum extract as an optional ingredient and moreover, the composition according to this invention may be used in personal care products such as creams or soap products and not as a drug.

US-A-20100092581 relates to an antibacterial composition comprising an extract from a plant belonging to the genus Arceuthobium, such as Arceuthobium Americanum. The composition shows activity against MRSA and may be used in a pharmaceutical composition or a disinfectant. Nonetheless, origanum is not listed in this document as potential ingredient.

A composition consisting of the extracts of horseradish and nasturtium is described in the Ärzte Zeitung (16 Dec. 2002). The bacteriostatic activity of said composition against MRSA is derived from the ingredient mustard oil; the composition, however, does not relate at all to origanum.

WO-A-2010049542 is directed to a hydrolyzate of at least one extract of at least one plant material, which shows antibacterial activity, inter alia against MRSA. Although a variety of potential plant materials listed in the document, extracts derived from origanum are not listed.

It is therefore the object of the present invention to provide a new and effective antibacterial composition for the treatment of bacterial infections. This object is attained by a composition for use in treating bacterial infections, comprising a concentrate or an extract of origanum in combination with at least one concentrate or extract of another plant.

The concentrate or extract of origanum according to the invention can be derived from the species. Origanum acutidens, Origanum amanum, Origanum calcaratum, Origanum compactum, Origanum dictamnus, Origanum laevigatum, Origanum leptocladum, Origanum libanoticum, Origanum majorana, Origanum microphyllum, Origanum rotundifolium, Origanum scabrum, Origanum sipyleum, Origanum syriacum, Origanum vulgare. The preferred origanum species according to the invention is Origanum vulgare.

In a preferred embodiment of the invention, in combination with any one of the embodiments listed above or below, the composition in accordance with the present invention is an extract of origanum in combination with at least one extract of another plant.

In a further preferred embodiment of the invention, in combination with any one of the embodiments listed above or below, the concentrate or extract of said composition for use is from the aboveground parts of the plant.

The term “plant” refers to all parts of the plant which are aboveground, including leaves, twigs, blossoms, fruits and seeds.

In a preferred embodiment, in combination with any one of the embodiments listed above or below, the plant of the composition for use is selected from the plants of the family Apiaceae, Armaranthaceae, Rosaceae, Grossulariaceae, Astereraceae, Cistaceae, Lamiaceae, Fabaceae and Elaeagnaceae.

In another preferred embodiment of the invention, in combination with any one of the embodiments listed above or below, the plant of the composition for use is selected from the genus Aegopodium, Salicornia, Chenopodium, Cydonia, Sorbus, Ribes, Cichorium, Cistus, Geum, Hippophaë, Sideritis, Cicer and Prunus.

In a more preferred embodiment of the invention, in combination with any one of the embodiments listed above or below, the plant of the composition for use is selected from Aegopodium podagraria, Chenopodium bonus-henricus, Sorbus terminalis, Ribes nigrum, Ribes rubrum, Cistus incanus, Cydonia oblonga, Cichorium intybus, Salicornia europaea, Geum urbanum, Hippophaë rhamnoides, Sideritis scardica, Cicer arietinum, and Prunus spinosa.

For preparing the concentrate or extract according to the present invention, all elements of the aboveground parts of the plant can be used. Preferably, the leaves, twigs and blossoms are used. Preferably, the plant material is coarsely cut.

According to the invention, the term “concentrate” is used representatively for all products that are obtained from a herbal subject by means of removal of water from the fresh plant.

According to the invention, the term “extract” is used representatively for all products that are obtained from a herbal subject by means of an extraction with a solvent, such as maceration or percolation.

As for the extraction, the aboveground parts of the plant are submitted either in the raw state or dried to maceration or percolation. In a preferred embodiment, the dried plant material is used.

The plant parts can be broken into small pieces in a suitable manner before the extraction, by means of rubbing or cutting them, for example. Alternatively, the plant parts can be pressed out directly after the harvest, meaning in the raw state, in order to produce a juice from pressing before the extraction.

Generally, an extraction of the plant parts including leaves, twigs and blossoms is performed with a suitable solvent. Suitable solvents are water, alcohols, such as methanol, ethanol or isopropyl alcohol, or chlorinated solvents, such as dichloromethane, as well as acetone, acetylacetone, ethylacetate, ammonia or glacial acetic acid, but also supercritical carbon dioxide. Mixtures of the solvents mentioned can also be used.

Furthermore, fats, such as pork fat, waxes, such as beeswax, or oils, such as olive oils and almond oil, can be used for the extraction. Preferably, almond oil is used.

In a preferred embodiment of the invention, in combination with any one of the embodiments listed above or below, the extract of the composition for use is an aqueous extract or an alcoholic extract. In a more preferred embodiment, in combination with any one of the embodiments listed below or above, water or a mixture of water with methanol or ethanol is used.

The extraction is normally carried out at temperatures between 25 and 100° C., depending on the boiling point of the solvent used. Preferred is an extraction at 95 to 100° C.

In another preferred embodiment of the invention, in combination with any one of the embodiments listed above or below, the extraction is normally carried out for 1 min to 8 h, more preferably 1 to 3 h, in particular the extraction is carried out for 1 h.

In order to achieve the highest possible yield, the plant material can be extracted a number of times. Preferably, the extraction is repeated 2 to 6 times, more preferably 3 times. In this case, it is also possible to use different solvents in the various extraction steps or an extraction with a solvent can be followed by an extraction with fat, wax or oil, or vice versa.

A maceration procedure is normally performed in 5 to 9 days, preferably for 7 days, at room temperature with a mixture of water and ethanol, by pouring the solvent mixture over the plant elements and letting this stand for the period of time mentioned.

According to the invention, a percolation of the plant parts is normally achieved by treating the parts with water at 95 to 100° C. for 4 to 5 hours by conducting the water through the plant parts.

The crude extraction product can also be concentrated and/or dried and/or further processed before use. To produce a dry extract, the solvent can be withdrawn from the liquid raw extract, the concentrated extract or the cleaned extract by, for example, spray drying, freeze drying or vacuum drying. The further processing can, for example, include cleaning steps known to the person skilled in the art, such as centrifugation, filtration and decantation, in order to remove suspended materials from the extract. Chromatography, such as column chromatography, gas chromatography or HPLC or steam distillation also is used for purification. In a preferred embodiment the crude product is used without further purification steps.

The composition for use according to the invention can be produced either by mixing the aboveground parts of Origanum and another plant before carrying out the extraction; alternatively and preferably, the concentrate or extract of Origanum is mixed with the extract of another plant so as to obtain the composition for use according to the invention.

In a preferred embodiment of the invention, in combination with any one of the embodiments listed above or below, the composition according to the invention comprises a concentrate or an extract of Origanum and another plant in a ratio of 1:10 to 10:1, preferably 1:5 to 5:1, and more preferably 1:2 to 2:1 and in particular 1:1.

The combinations of Origanum with other plant comprise Origanum/Apiaceae, Origanum/Armaranthaceae, Origanum/Rosaceae, Origanum/Grossulariaceae, Origanum/Astereraceae, Origanum/Cistaceae, Origanum/Lamiaceae, Origanum/Fabaceae and Origanum/Elaeagnaceae. Preferred are combinations of Origanum/Aegopodium, Origanum/Salicornia, Origanum/Chenopodium, Origanum/Cydonia, Origanum/Sorbus, Origanum/Ribes, Origanum/Cichorium, Origanum/Cistus, Origanum/Geum, Origanum/Hippophae, Origanum/Sideritis, Origanum/Cicer, and Origanum/Prunus. More preferred are combinations of Origanum/Aegopodium podagraria, Origanum/Chenopodium bonus-henricus, Origanum/Sorbus terminalis, Origanum/Ribes nigrum, Origanum/Ribes rubrum, Origanum/Cistus incanus, Origanum/Cydonia oblonga, Origanum/Cichorium intybus, Origanum/Salicornia eurpaea, Origanum/Geum urbanum, Origanum/Hippophaë rhamnoides, Origanum/Sideritis scardica, Origanum/Cicer arietinum, Origanum/Prunus spinosa. In particular preferred are combinations of Origanum vulgare/Cistus incanus, Origanum vulgare/Ribus nigrum, Origanum vulgare/Cydonia oblonga, and Origanum vulgare/Aegopodium podagraria.

In a preferred embodiment in combination with any of the above or below embodiments, the composition according to the invention moreover comprises combinations of Origanum and two or more other plants. Preferred embodiments are Origanum/Cistus/Ribes and Origanum/Cydonia/Aegopodium, in particular Origanum vulgare/Cistus incanus/Ribes nigrum and Origanum vulgare/Cydonia oblonga/Aegopodium podagraria

In a preferred embodiment of the invention, in combination with any one of the embodiments listed above or below, the composition according to the invention is in liquid, dry or semi-solid form.

In another preferred embodiment of the invention, in combination with any one of the embodiments listed above or below, the bacterial infection is caused by pathogenous bacteria.

The term “pathogenous bacteria” as used herein refers to bacteria that cause bacterial infections. Pathogenous bacteria include both Gram-positive and Gram-negative bacteria, forming small coccobacilli; small, round, ovoid rods; large, blunt-ended rods; small, slender, pleomorphic rods; ovoid to spherical rods; long, slender, flexible, spiral- or corkscrew-shaped rods; slender, short rods; curved rods with single polar flagellum, and Kidney bean-shaped rods.

In a preferred embodiment of the invention, in combination with any one of the embodiments listed above or below, the bacterial infection is an infection of the gastrointestinal tract, an infection of the urogenital tract, an infection of the respiratory tract, like, for example rhinitis, tonsillitis, pharyngitis, bronchitis, pneumonia, an infection of the inner organs, like, for example, nephritis, hepatitis, peritonitis, endocarditis, meningitis, osteomyelitis, an infection of the eyes, the ears as well as a cutaneous and a subcutaneous infection, diarrhea, skin disorders, toxic shock syndrome, bacteraemia, sepsis, and tuberculosis.

In yet another preferred embodiment of the invention, in combination with any one of the embodiments listed above or below, the pathogenous bacteria are selected from the genus Staphylococcus, Streptococcus, Pseudomonas, Escherichia, Salmonella, Helicobacter, Neisseria, Campylobacter, Chlamydia, Clostridium, Vibrio, Triponema, Mycobacterium, Klebsiella, Actinomyces, Bacterioides, Bordetella, Borrelia, Brucella, Corynebacterium, Diplococcus, Enterobacter, Fusobacterium, Leptospira, Listeria, Pasteurella, Proteus, Rickettsia, Shigella, Sphaerophorus, Yersinia, or combinations thereof.

In a further preferred embodiment of the invention, in combination with any one of the embodiments listed above or below, the bacteria are selected from Streptococcus mutans, Salmonella typhimurium, Pseudomonas aeruginosa, and Yersinia enterocolitica.

In a further preferred embodiment of the invention, in combination with any one of the embodiments listed above or below, the bacteria are antibiotic-resistant bacteria. “Antibiotic-resistant” as used herein means that neither a bactericide nor a bacteriostatic effect is achieved by common antibiotics like β-lactam antibiotics, tetracyclines, aminoglycosides, macrolide antibiotics, lincosamids, gyrase inhibitors (chinolones), sulfonamids and trimethoprime, glycopepide antibiotics, polypeptide antibiotics and nitroimidazole derivatives when the antibiotic is applied to the bacteria.

In another preferred embodiment, in combination with any one of the embodiments listed above or below, the antibiotic-resistant bacteria represent methicillin-resistant Staphylococcus aureus. The term methicillin-resistant is used herein synonymously to “multiresistant” and “vancomycin-resistant”.

In a preferred embodiment of the invention, in combination with any one of the embodiments listed above or below, the composition for use is administrated orally, intranasally or topically.

In another preferred embodiment, in combination with any one of the embodiments listed above or below, the composition for use is in the form of a nasal agent, inhalation mixture, aerosol, mouthwash, mouth spray, nose spray or room spray. More preferably, the composition is in form of a mouth spray, a nose spray or a room spray, in particular in form of a nose spray.

In a more preferred embodiment, the composition is in the form of an aerosol or room spray. Preferably a liquid or solid composition according to the invention is used for this. In addition to the extract, the aerosol or room spray can also contain pharmaceutically harmless substances, carrier media and auxiliary agents. The aerosol or room spay can be used for disinfecting objects and rooms with which bacteria come into contact or could potentially come into contact, particularly means of transport of all types in which people, animals and/or foodstuffs are transported. For example, an airplane can be sprayed with the aerosol according to the invention or with the room spray according to the invention before takeoff, in order to prevent the spread of the viruses and consequently to minimize the risk of infection for people. The aerosol or room spray can also be sprayed in the presence of people, e.g. in waiting rooms, because it does not cause any toxic effects whatsoever in people.

In a particularly preferred embodiment, in combination with any one of the embodiments listed above or below, the composition is applied as a nose spray, particularly for the treatment of bacteria induced diseases of the respiratory tract and sinuses. It is preferable for the composition to be in the form of a liquid extract in this case.

In another preferred embodiment, the composition for use according to the invention, in combination with any one of the embodiments listed above is in the form of a tablet, coated tablet, effervescent tablet, capsule, powder, granulate, sugar-coated tablet, lozenge, pill, ampoule, drop, suppository, emulsions, ointments, gels, tinctures, pastes, creams, moist compresses, gargling solution or plant juice.

As for the oral application, in combination with any one of the embodiments listed above or below, the composition is preferably administered in the form of a tablet. It is preferable for the composition to be in the form of a dry extract in this case.

In another preferred embodiment, in combination with any one of the embodiments listed above or below, the composition can be used according to the invention as a solution, in particular, a gargling solution, mouthwash or tincture, particularly for the treatment of bacterial infections of mouth and upper throat.

Suppositories, in combination with any one of the embodiments listed above or below, represent a preferred embodiment for rectal and vaginal administration of the composition according to the invention.

As for the topical administration, the composition for use according to the invention is administered in the form of emulsions, ointments, gels, tinctures, pastes, creams or moist compresses. In this case, the composition is preferably used in the form of an extract in which the active substances are withdrawn from the plant by means of extraction with a fat, wax or oil. It is furthermore preferred for this extract to be further processed into a dry extract, which is subsequently mixed with or dissolved in a fat, wax or oil.

The concentration of the composition in the application form varies, depending on the type of application. As a rule, the quantity of the composition amounts to between 0.5 and 1,000 mg per dosing unit for solid application forms. Preferably the quantity of the composition amounts to between 1 and 500 mg per unit. In liquid application forms, the composition can be in a concentration of 1 μg/ml to 100 mg/ml, preferably from 25 μg/ml to 50 mg/ml. In the case of semi-solid application forms, the content of the composition amounts to 1 to 90% by weight, preferably 5 to 75% by weight.

Further elements, such as vitamins and minerals, can be added to the composition used according to the invention.

The composition can, for example, also be added to animal feed or foodstuffs, such as drinks. In the form of an extract, the composition itself can also be infused as tea. It is also possible, however, for hot water to be poured directly over the plant parts, for example, the leaves of the origanum plants, for tea preparation. Furthermore, the composition can be a constituent of food supplements, whose ingestion in the winter months can contribute to strengthening the body's defenses and to treat a bacterial infection.

The following examples explain the present invention.

General Procedure for the Production of Liquid and Dry Extracts:

The collected plant material is visually checked, and non-original, damaged or eaten away parts are removed.

The purified material is spread on a table in a green house and covered with paper for drying. The plant parts are turned around on a daily basis and visually checked for non-original and damaged parts, which are removed. The residual moisture of the plant material is determined on regular basis. The material is good for further processing when the residual water content is at a maximum of 10%.

The plant material is coarsely cut and transferred to a beaker; Cold, distilled water is added (10 to 30-times the quantity of the plant material). The resulting mixture is heated on a hotplate while being stirred until it starts boiling. Simmering is continued for 1 h.

The hot liquid is strained, and the residual plant material is squeezed out. The resulting aqueous extract is filled directly in a bottle.

For the production of the dry extract, the liquid extract is filled in metal bowls and concentrated at 80° C. in a drying oven until the solvent has completely evaporated. The residue is scraped out of the metal bowl and weighed. Alternatively, the liquid extract is freeze-dried according to standard procedures known to the person skilled in the art.

Preparation of an Extract of Origanum

All above-ground parts of Origanum vulgare are used for the extraction.

Following the general procedure, 150 g of coarsely cut Origanum vulgare is heated in 1500 ml of distilled water for 1 h. The resulting aqueous extract is filled directly in a bottle.

Preparation of an Extract of Ribes nigrum

The leaves of Ribes nigrum are used for the extraction.

Following the general procedure, 26.7 g of coarsely cut Ribes nigrum leaves are heated in 800 ml of distilled water for 1 h. The resulting aqueous extract is filled directly in a bottle.

Preparation of an extract of Cistus incanus

Blossoms and little twigs of Cistus incanus are used for the extraction.

Following the general procedure, 100 g of coarsely cut Cistus incanus material is heated in 1500 ml of distilled water for 1 h. The resulting aqueous extract is filled directly in a bottle.

Antibacterial Effect of Origanum vulgare, Ribes nigrum and Cistus incanus Against Staphylococcus aureus

Origanum vulgare (Test Substance A)

Formulation: 5 mg/ml in H₂O; substance solid (dry frozen)

Test System:

Test model: Staphylococcus aureus (USA300=MRSA) on Mueller-Hinton agar (MH-agar)

Test groups: Substance A (5 mg)

Procedure: The dry-frozen extract was dissolved in water. 100 μl of MRSA solution was plated on MH agar. Subsequently, holes of 8 mm where punched out of the MH agar and filled with 100 μl extract solution. After 22 h at 37° C., the holes were checked optically for inhibition of growth.

Application of MRSA bacteria: 10⁻³/1.63×10⁵/100 μl/agar plate

Overnight MRSA culture: 10 μl in 8 ml TBS medium, 22 h, 37° C., shake

Ribes nigrum (Test Substance B)

Formulation: 5 mg/ml in H₂O; substance solid (dry frozen)

Test System:

Test model: Staphylococcus aureus (USA300=MRSA) on Mueller-Hinton agar (MH-agar)

Test groups: Substance B (5 mg)

Procedure: The dry-frozen extract was dissolved in water. 100 μl of MRSA solution was plated on MH agar. Subsequently, holes of 8 mm where punched out of the MH agar and filled with 100 μl extract solution. After 22 h at 37° C., the holes were checked optically for inhibition of growth.

Application of MRSA bacteria: 10⁻³/1.63×10⁵/100 μl/agar plate

Overnight MRSA culture: 10 μl in 8 ml TBS medium, 22 h, 37° C., shake

Cistus incanus (Test Substance C)

Formulation: 2.5 mg/ml in H₂O; substance solid (dry frozen)

Test System:

Test model: Staphylococcus aureus (USA300=MRSA) on Mueller-Hinton agar (MH-agar)

Test groups: Substance C (2.5 mg)

Procedure: The dry-frozen extract was dissolved in water. 100 μl of MRSA solution was plated on MH agar. Subsequently, holes of 8 mm where punched out of the MH agar and filled with 100 μl extract solution. After 22 h at 37° C., the holes were checked optically for inhibition of growth.

Application of MRSA bacteria: 10⁻³/1.63×10⁵/100 μl/agar plate

Overnight MRSA culture: 10 μl in 8 ml TBS medium, 22 h, 37° C., shake

Synergystic Antibacterial Effect of Origanum vulgare and Ribes nigrum Against Staphylococcus aureus

Test Objects:

Test substances: A (=Origanum vulgare)+B (=Ribes nigrum)

Formulation: Substance A: 5 mg/ml in H₂O; substance solid (dry frozen); Substance B: 5 mg/ml in H₂O; substance solid (dry frozen)

Test System:

Test model: Staphylococcus aureus (USA300=MRSA) on Mueller-Hinton agar (MH-agar)

Test groups: Substance A (5 mg)+Substance B (5 mg)

Procedure: The dry-frozen substances were dissolved in water. 100 μl of MRSA solution was plated on MH agar. Subsequently, holes of 8 mm where punched out of the MH agar and filled with 100 μl substance solution. After 22 h at 37° C., the holes were checked optically for inhibition of growth.

Application of MRSA bacteria: 10⁻³/1.63×10⁵/100 μl/agar plate

Overnight MRSA culture: 10 μl in 8 ml TBS medium, 22 h, 37° C., shake

Result:

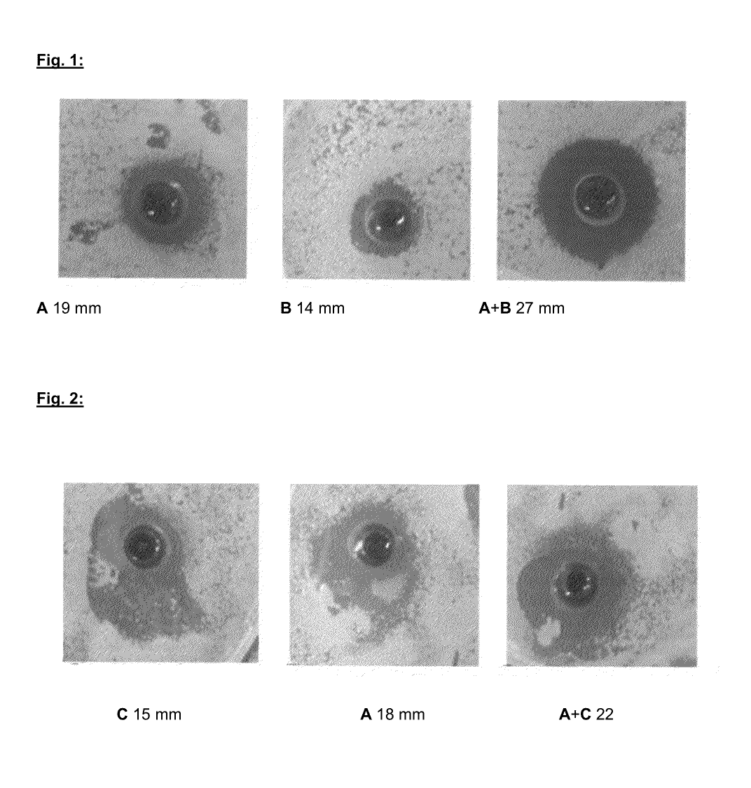

As can be seen from FIG. 1, a better inhibition of the growth of MRSA is obtained when using the combination of Origanum vulgare and Ribes nigrum.

Synergystic Antibacterial Effect of Origanum vulgare and Cistus incanus Against Staphylococcus aureus

Test substances: A (=Origanum vulgare)+C (=Cistus)

Formulation: Substance A: 5 mg/ml in H₂O; substance solid (dry frozen); Substance C: 2.5 mg/ml in H₂O; substance solid (dry frozen)

Test System:

Test model: Staphylococcus aureus (USA300=MRSA) on Mueller-Hinton agar (MH-agar)

Test groups: Substance A (5 mg)+Substance C (2.5 mg)

Procedure: The dry-frozen substances were dissolved in water. 100 μl of MRSA solution was plated on MH agar. Subsequently, holes of 8 mm where punched out of the MH agar and filled with 100 μl substance solution. After 22 h at 37° C., the holes were checked optically for inhibition of growth.

Application of MRSA bacteria: 10⁻³/1.63×10⁵/100 μl/agar plate

Overnight MRSA culture: 10 μl in 8 ml TBS medium, 22 h, 37° C., shake

Result:

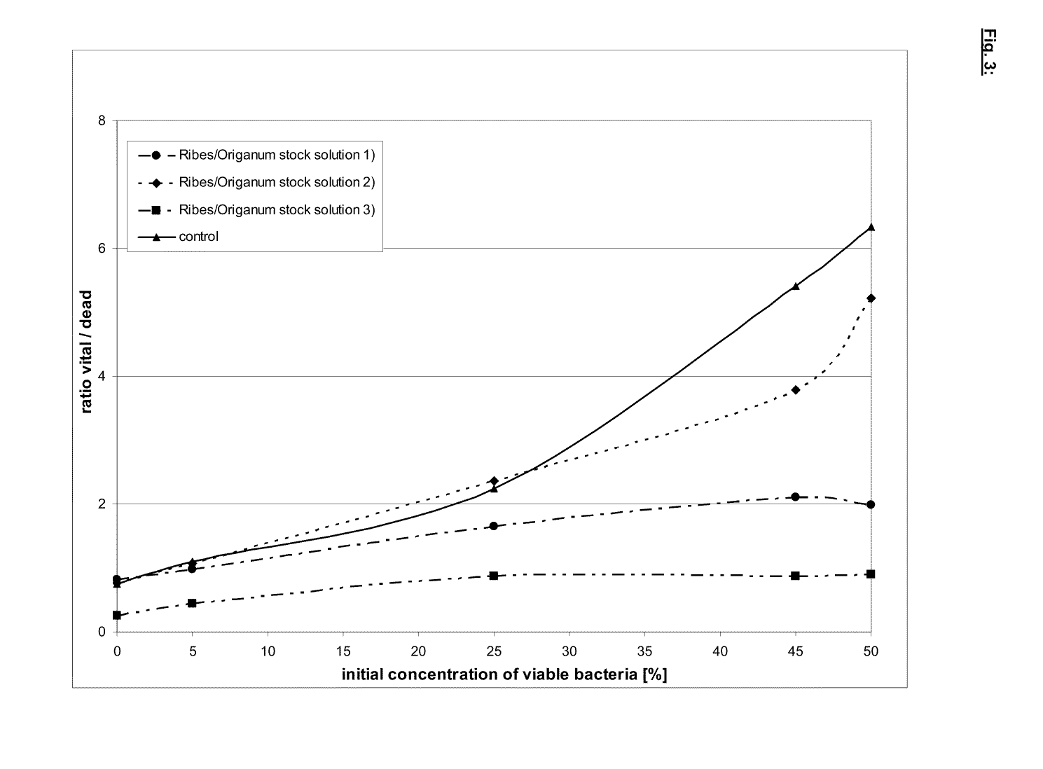

As can be seen from FIG. 2, a better inhibition of the growth of MRSA is obtained when using the combination of Origanum vulgare and Cistus incanus.

Antibacterial Effect of Origanum vulgare and Ribes nigrum Against Streptococcus mutans

Preparation of a Liquid Extract of Origanum and Ribes nigrum

All above-ground parts of Origanum vulgare and the leaves of Ribes nigrum are used for the extraction.

Following the general procedure, 70 g of coarsely cut Origanum vulgare and 30 g of coarsely cut Ribes nigrum leaves are boiled in 1000 ml of distilled water for 1 h. The resulting aqueous extract is filtered and boiled again for 5 min. The resulting extract is filled directly in a bottle. The concentration of the liquid extract is 30.5 mg/ml.

Preparation of a Dry Extract of Origanum vulgare

All above-ground parts of Origanum vulgare are used for the extraction.

Following the general procedure, 150 g of coarsely cut Origanum vulgare is heated in 1500 ml of distilled water for 1 h. The resulting aqueous extract is dried according to standard procedures known to the person skilled in the art. The content of dry substance is at least 92% by weight, based on the total weight of the dry extract.

Preparation of a Dry Extract of Ribes nigrum

The leaves of Ribes nigrum are used for the extraction.

Following the general procedure, 26.7 g of coarsely cut Ribes nigrum leaves are heated in 800 ml of distilled water for 1 h. The resulting aqueous extract is dried according to standard procedures known to the person skilled in the art. The content of dry substance is at least 92% by weight, based on the total weight of the dry extract.

Preparation of a Dry Extract of Origanum vulgare and Ribes nigrum

The dry extract of Origanum vulgare is mixed thoroughly with the dry extract of Ribes nigrum in a weight ratio of 1:1.

Procedure:

BacLight Viability Assay, In Vitro Experiment

The LIVE/DEAD BacLight Bacterial Viability Kit (Invitrogen, Molecular probes, Darmstadt, Germany) adopts two nucleic acid stains—green-fluorescent SYTO 9 stain and red-fluorescent propidium iodide stain. For vital cells, the small molecule SYTO 9 is used penetrating vital and dead (avital) cells, whereas the counterstain propidium iodide stains only dead cells.

The assays were carried out according to manufacturer's instructions and the fluorescence intensity was measured in a 96-well plate reader. A suspension of Streptococcus mutans in saline solution was prepared after cultivation overnight; 50% of the bacteria were inactivated with heat (1 h; 95° C.).

The above-described dry extract of Origanum vulgare and Ribes nigrum was dissolved in distilled water with use of ultrasound. The liquid extract of Origanum vulgare and Ribes nigrum was used directly without further dilution. The following stock solutions were prepared and tested for antibacterial activity against Streptococcus mutans:
1) 8 mg/ml of Origanum vulgare and 8 mg/ml of Ribes nigrum (from above-described dry extract)
2) 0.8 mg/ml of Origanum vulgare and 0.8 mg/ml of Ribes nigrum (from above-described dry extract)
3) 30.5 mg/ml (from above-described liquid extract).

The vital bacteria were mixed 1:1 with each of the stock solutions and incubated for 10 min. Afterwards, these suspensions were mixed with heat-inactivated bacteria (0:100; 5:95; 25:75; 45:55; 50:50). A volume of 0.5 μl of BacLight-staining solution (component A and B 1:1) was added to 250 μl of these mixtures. The staining was incubated for 10 min in a dark chamber. A volume of 100 μl from each sample was pipetted in a micro titer plate, and the fluorescence was measured. The excitation wavelength was 470 nm; emission was recorded at 530 nm for the vital and at 620 nm for the avital cells. The measurements were carried out in duplicates to equalize inhomogenities of the suspension. For evaluation of the recorded data, the ratio of vital and dead (avital) cells was calculated according to ratio=emission vital/emission dead bacteria. Experiments with saline solution served as a reference/negative control.

Result:

As can be seen from FIG. 3, the extracts of Origanum vulgare and Ribes nigrum show an antibacterial activity against Streptococcus mutans.

Preparation of a Liquid Extract of Origanum vulgare and Ribes nigrum

All above-ground parts of Origanum vulgare and the leaves of Ribes nigrum are used for the extraction.

Following the general procedure, 70 g of coarsely cut Origanum vulgare and 30 g of coarsely cut Ribes nigrum leaves are boiled in 1000 ml of distilled water for 1 h. The resulting aqueous extract is filtered and boiled again for 5 min. The resulting extract is filled directly in a bottle. The concentration of the pure liquid extract is 30.5 mg/ml. The extract is further diluted with LB medium in the given ratios.

Inhibition Zone Assay of Staphylococcus aureus

Test System:

Overnight MRSA culture: 10 μl in 8 ml TBS medium, 22 h, 37° C., shake

Test model: Staphylococcus aureus (USA300=MRSA) on Mueller-Hinton agar (MH-agar)

Test groups: above-described liquid extract of Origanum vulgare and Ribes nigrum; pure, 1:2, and 1:4 dilution

Procedure: 100 μl of MRSA solution was plated on MH agar. Subsequently, holes of 8 mm where punched out of the MH agar and filled with 100 μl of the liquid extracts in the given concentrations. After 22 h at 37° C., the holes were checked optically for inhibition of growth.

Application of MRSA bacteria: 10⁻³/1.63×10⁵/100 μl/agar plate

Result:

An inhibition of the growth of MRSA was observed. When using the pure extract of Origanum vulgare and Ribes nigrum an inhibition zone of 28 mm on the MH agar was observed. With a dilution of 1:2 (extract:LB medium) the inhibition zone was 19 mm, with a dilution of 1:4 (extract:LB medium) the inhibition zone was 13 mm.

Inhibition Zone Assay of Salmonella typhimurium

Test System:

Overnight Salmonella typhimurium culture: 10 μl in 8 ml LB medium, 22 h, 37° C., shake, diluted 1:100 in LB medium

Test model: Salmonella typhimurium on Mueller-Hinton agar (MH-agar)

Test groups: above-described liquid extract of Origanum vulgare and Ribes nigrum; pure

Procedure: 100 μl of Salmonella typhimurium solution was plated on MH agar. Subsequently, holes of 8 mm where punched out of the MH agar and filled with 100 μl of the liquid extract in the given concentration. After 22 h at 37° C., the holes were checked optically for inhibition of growth.

Result:

An inhibition of the growth of Salmonella typhimurium was observed. An inhibition zone of 10 mm on the MH agar was observed.

Inhibition Zone Assay of Yersinia enterocolitica

Test System:

Overnight Yersinia enterocolitica culture: 10 μl in 8 ml LB medium, 22 h, 27° C., shake, diluted 1:1000 in LB medium

Test model: Yersinia enterocolitica on Mueller-Hinton agar (MH-agar)

Test groups: above-described liquid extract of Origanum vulgare and Ribes nigrum; pure, 1:2 dilution

Procedure: 100 μl of Yersinia enterocolitica solution was plated on MH agar. Subsequently, holes of 8 mm where punched out of the MH agar and filled with 100 μl of the liquid extracts in the given concentrations. After 22 h at 27° C., the holes were checked optically for inhibition of growth.

Result:

An inhibition of the growth of Yersinia enterocolitica was observed. When using the pure extract of Origanum vulgare and Ribes nigrum an inhibition zone of 14 mm on the MH agar was observed. With a dilution of 1:2 (extract:LB medium), the inhibition zone was 10 mm.

Growth Curves of Salmonella typhimurium with Liquid Extract

Test System:

Overnight Salmonella typhimurium culture: 10 μl in 8 ml LB medium, 22 h, 37° C., shake

Test model: Salmonella typhimurium on Mueller-Hinton agar (MH-agar)

10 ml mixtures were prepared as follows:
a) LB medium
b) LB medium+50 μl Salmonella typhimurium
c) LB medium+above-described liquid extract of Origanum vulgare and Ribes nigrum (4:1)
d) LB medium+above-described liquid extract of Origanum vulgare and Ribes nigrum (4:1)+50 μl Salmonella typhimurium

Procedure: 50 μl of these mixtures were diluted with 450 μl LB medium in photometric cuvettes, OD measurements were carried out after 0 h, 1 h, 2 h, 3 h, 4 h, 5 h, 6 h, 7 h, 8 h, 24 h, 48 h at 600 nm.

Result:

The liquid extract of Origanum vulgare and Ribes nigrum strongly inhibits the growth of Salmonella typhimurium for a long time, even more than 48 h.

Growth Curves of Pseudomonas aeruginosa with Liquid Extract

Test System:

Overnight Pseudomonas aeruginosa culture: 10 μl in 8 ml LB medium, 22 h, 37° C., shake

10 ml mixtures were prepared as follows:
a) LB medium
b) LB medium+50 μl Pseudomonas aeruginosa
c) LB medium+above-described liquid extract of Origanum vulgare and Ribes nigrum (2:1)
d) LB medium+above-described liquid extract of Origanum vulgare and Ribes nigrum (4:1)
e) LB medium+above-described liquid extract of Origanum vulgare and Ribes nigrum (2:1)+50 μl Pseudomonas aeruginosa
f) LB medium+above-described liquid extract of Origanum vulgare and Ribes nigrum (4:1)+50 μl Pseudomonas aeruginosa

Procedure: 100 μl of these mixtures were diluted with 900 μl LB medium in photometric cuvettes, OD measurements were carried out after 0 h, 1 h, 2 h, 3 h, 4 h, 5 h, 6 h, 7 h, 8 h, 24 h, 48 h at 600 nm.

Result:

If the liquid extract of Origanum vulgare and Ribes nigrum is used in a dilution of 1:2, it inhibits the growth of Pseudomonas aeruginosa for approximately 8 h. At a dilution of 1:4, the extract strongly inhibits bacterial growth after 8 h.

Growth Curves of Yersinia enterocolitica with Liquid Extract

Test System:

Overnight Yersinia enterocolitica culture: 10 μl in 8 ml LB medium, 22 h, 27° C., shake

10 ml mixtures were prepared as follows:
a) LB medium
b) LB medium+50 μl Yersinia enterocolitica
c) LB medium+above-described liquid extract of Origanum vulgare and Ribes nigrum (2:1)
d) LB medium+above-described liquid extract of Origanum vulgare and Ribes nigrum (4:1)
e) LB medium+above-described liquid extract of Origanum vulgare and Ribes nigrum (2:1)+50 μl Yersinia enterocolitica
f) LB medium+above-described liquid extract of Origanum vulgare and Ribes nigrum (4:1)+50 μl Yersinia enterocolitica

Procedure: 100 μl of these mixtures were diluted with 900 μl LB medium in photometric cuvettes, OD measurements were carried out after 0 h, 1 h, 2 h, 3 h, 4 h, 5 h, 6 h, 7 h, 8 h, 24 h, 48 h at 600 nm.

Result:

The liquid extract of Origanum vulgare and Ribes nigrum inhibits the growth of Yersinia enterocolitica for approximately 8 h in both dilutions.

MIC₅₀ Assay

The MIC₅₀ value describes the Minimum Inhibitory Concentration required to inhibit the growth of 50% of organisms.

MIC₅₀ Assay of Salmonella typhimurium

Test System:

Overnight Salmonella typhimurium culture: 10 μl in 8 ml LB medium, 22 h, 37° C., shake

Test groups: above-described liquid extract of Origanum vulgare and Ribes nigrum; pure, 1:2, 1:4, 1:8, 1:16, 1:32, 1:64, and 1:128 (diluted in LB medium)

Procedure: 9 ml of LB medium was inoculated with 1 ml of the overnight culture and incubated at 37° C. in a shaker until an OD₆₀₀ between 0.5 and 0.6 was measured with a photometer. The bacteria was counted with a Neubauer Zählkammer and diluted to 25×10⁵/ml medium. 2 ml of the above-described liquid extract of Origanum vulgare and Ribes nigrum, pure, 1:2, 1:4, 1:8, 1:16, 1:32, 1:64, and 1:128 in LB medium was inoculated with 200 μl (approximately 5×10⁵) Salmonella typhimurium. Dilutions of the liquid extracts without bacteria as well as 2 ml LB medium or medium with bacteria served as controls. Incubation for 19 h at 37° C. in a shaker. The mixtures were diluted 1:10 in photometric cuvettes and the OD was determined at 600 nm.

Result:

The average dilution where the MIC₅₀ is reached is 1:9 (extract:LB medium).

MIC₅₀ Assay of Pseudomonas aeruginosa

Test System:

Overnight Pseudomonas aeruginosa culture: 10 μl in 8 ml LB medium, 22 h, 37° C., shake

Test groups: above-described liquid extract of Origanum vulgare and Ribes nigrum; pure, 1:2, 1:4, 1:8, 1:16, 1:32, 1:64, 1:128, 1:256, 1:512 (diluted in LB medium)

Procedure: 9 ml of LB medium was inoculated with 1 ml of the overnight culture and incubated at 37° C. in a shaker until an OD₆₀₀ between 0.5 and 0.6 was measured with a photometer. The bacteria was counted with a Neubauer Zählkammer and diluted to 25×10⁵/ml medium. 2 ml of the above-described liquid extract of Origanum vulgare and Ribes nigrum, pure, 1:2, 1:4, 1:8, 1:16, 1:32, 1:64, 1:128, 1:256, 1:512 in LB medium was inoculated with 200 μl (approximately 5×10⁵) Pseudomonas aeruginosa. Dilutions of the liquid extracts without bacteria as well as 2 ml LB medium or medium with bacteria served as controls. Incubation for 19 h at 37° C. in a shaker. The mixtures were diluted 1:10 in photometric cuvettes and the OD was determined at 600 nm.

Result:

The average dilution where the MIC₅₀ is reached is 1:70 (extract:LB medium).

MIC₅₀ Assay of Yersinia enterocolitica

Test System:

Overnight Yersinia enterocolitica culture: 10 μl in 8 ml LB medium, 22 h, 27° C., shake

Test groups: above-described liquid extract of Origanum vulgare and Ribes nigrum; pure, 1:2, 1:4, 1:8, 1:16, 1:32, 1:64, 1:128, 1:256, 1:512 (diluted in LB medium)

Procedure: 9 ml of LB medium was inoculated with 1 ml of the overnight culture and incubated at 37° C. in a shaker until an OD₆₀₀ between 0.5 and 0.6 was measured with a photometer. The bacteria was counted with a Neubauer Zählkammer and diluted to 25×10⁵/ml medium. 2 ml of the above-described liquid extract of Origanum vulgare and Ribes nigrum, pure, 1:2, 1:4, 1:8, 1:16, 1:32, 1:64, 1:128, 1:256, 1:512 in LB medium was inoculated with 200 μl (approximately 5×10⁵) Yersinia enterocolitica. Dilutions of the liquid extracts without bacteria as well as 2 ml LB medium or medium with bacteria served as controls. Incubation for 19 h at 27° C. in a shaker. The mixtures were diluted 1:10 in photometric cuvettes and the OD was determined at 600 nm.

Result:

The average dilution where the MIC₅₀ is reached is 1:50 (extract:LB medium).

MIC₅₀ Assay of Staphylococcus aureus

Test System:

Overnight MRSA culture: 10 μl in 8 ml LB medium, 22 h, 37° C., shake

Test groups: above-described liquid extract of Origanum vulgare and Ribes nigrum; pure, 1:2, 1:4, 1:8, 1:16, 1:32, 1:64, 1:128, 1:256, 1:512 (diluted in LB medium)

Procedure: 9 ml of LB medium was inoculated with 1 ml of the overnight culture and incubated at 37° C. in a shaker until an OD₆₀₀ between 0.5 and 0.6 was measured with a photometer. The bacteria was counted with a Neubauer Zählkammer and diluted to 25×10⁵/ml medium. 2 ml of the above-described liquid extract of Origanum vulgare and Ribes nigrum, pure, 1:2, 1:4, 1:8, 1:16, 1:32, 1:64, 1:128, 1:256, 1:512 in LB medium was inoculated with 200 μl (approximately 5×10⁵) Staphylococcus aureus. Dilutions of the liquid extracts without bacteria as well as 2 ml LB medium or medium with bacteria served as controls. Incubation for 19 h at 37° C. in a shaker. The mixtures were diluted 1:10 in photometric cuvettes and the OD was determined at 600 nm.

Result:

The average dilution where the MIC₅₀ is reached is 1:73 (extract:LB medium).

1. Composition for use in treating bacterial infections, comprising a concentrate or an extract of origanum in combination with at least one concentrate or extract of another plant.

2. The composition for use according to claim 1, wherein the concentrate or extract is from the aboveground parts of the plant.

3. The composition for use according to claim 1, wherein the plant is selected from plants of the family Apiaceae, Armaranthaceae, Rosaceae, Grossulariaceae, Asteraceae, Cistaceae, Lamiaceae, Fabaceae, and Elaeagnaceae.

4. The composition for use according to claim 3, wherein the plant is selected from the genus Aegopodium, Salicornia, Chenopodium, Cydonia, Sorbus, Ribes, Cichorium, Cistus, Geum, Sideritis, Cicer, Hippophae and Prunus.

5. The composition for use according to claim 4, wherein the edible plant is selected from Aegopodium podagraria, Chenopodium bonus-henricus, Sorbus terminalis, Ribes nigrum, Ribes rubrum, Cistus incanus, Cydonia oblonga, Cichorium intybus, Salicornia europaea, Geum urbanum, Hippophaë rhamnoides, Sideritis scardica, Cicer arietinum, and Prunus spinosa.

6. The composition for use according to claim 1, wherein the bacterial infection is caused by pathogenous bacteria.

7. The composition for use according to claim 6, wherein the pathogenous bacteria are selected from the genus Staphylococcus, Streptococcus, Pseudomonas, Escherichia, Salmonella, Helicobacter, Neisseria, Campylobacter, Chlamydia, Clostridium, Vibrio, Triponema, Mycobacterium, Klebsiella, Actinomyces, Bacterioides, Bordetella, Borrelia, Brucella, Corynebacterium, Diplococcus, Enterobacter, Fusobacterium, Leptospira, Listeria, Pasteurella, Proteus, Rickettsia, Shigella, Sphaerophorus, Yersinia, or combinations thereof.

8. The composition for use according to claim 6, wherein the bacteria are antibiotic-resistant bacteria.

9. The composition for use according to claim 8, wherein the antibiotic-resistant bacteria are Methicillin-resistant Staphylococcus aureus.

10. The composition for use according to claim 1, wherein the composition is in liquid, dry or semi-solid form.

11. The composition for use according to claim 1, wherein the extract is an aqueous extract or an alcoholic extract.

12. The composition for use according to claim 1, wherein the composition is administered orally, intranasally or topically.

13. The composition for use according to claim 1, wherein the composition is in the form of a nasal agent, inhalation mixture, aerosol, mouthwash, mouth spray, nose spray or room spray.

14. The composition for use according to claim 1, wherein the composition is in the form of a tablet, coated tablet, effervescent tablet, capsule, powder, granulate, sugar-coated tablet, lozenge, pill, ampoule, drop, suppository, emulsions, ointments, gels, tinctures, pastes, creams, moist compresses, gargling solution or plant juice.

15. The composition for use according to claim 1, wherein the bacterial infection is an infection of the gastrointestinal tract, an infection of the urogenital tract, an infection of the respiratory tract, including rhinitis, tonsillitis, pharyngitis, bronchitis, pneumonia, an infection of the inner organs, including nephritis, hepatitis, peritonitis, endocarditis, meningitis, osteomyelitis, an infection of the eyes, the ears as well as a cutaneous and a subcutaneous infection, diarrhea, skin disorders, toxic shock syndrome, bacteraemia, sepsis, and tuberculosis.

16. The composition for use according to claim 1, wherein the bacteria are selected from Streptococcus mutans, Salmonella typhimurium, Pseudomonas aeruginosa, and Yersinia enterocolitica.

17. A method for treatment of a bacterial infection, comprising administering to a patient in need of said treatment a concentrate or an extract of origanum in combination with at least one concentrate or extract of another plant.

18. The method of claim 17, wherein the concentrate or extract is from the aboveground parts of the plant.

19. The method of claim 17, wherein the plant is selected from plants of the family Apiaceae, Armaranthaceae, Rosaceae, Grossulariaceae, Asteraceae, Cistaceae, Lamiaceae, Fabaceae, and Elaeagnaceae.

20. The method of claim 17, wherein the plant is selected from the genus Aegopodium, Salicornia, Chenopodium, Cydonia, Sorbus, Ribes, Cichorium, Cistus, Geum, Sideritis, Cicer, Hippophae and Prunus.
